(19) United States
(12) Patent Application Publication
(10) Pub. No.: US 2010/0159577 A1
(43) Pub. Date: Jun. 24, 2010

(54) AUTOMATIC CULTURE DEVICE, SLIDE VALVE, AND DISPOSABLE CONTAINER

(75) Inventor: Tomoyoshi TOKUMARU, Hirakata City (JP)

Correspondence Address: MOTS LAW PLLC, Suite 602, 1629 K Street, N.W., Washington, DC, 20006, US

(73) Assignee: SANYO ELECTRIC CO., LTD, Moriguchi City (JP)

(21) Appl. No.: 12/640,402

(22) Filed: Dec. 17, 2009

(30) Foreign Application Priority Data
Dec. 24, 2008 (JP) 2008-328108

(51) Int. Cl. C12M 1/00

(52) U.S. Cl. 435/289.1

(57) ABSTRACT

A culture device includes: a housing accommodating therein a preservation container preserving a first solution liquid for cell culture, the housing having an opening for discharging the first solution liquid out of the housing; a cultivation container having an opening, in which the cell culture can be performed; and a slide valve provided between the housing and the cultivation container. The slide valve includes: a slide valve body; a connection hole formed in the slide valve body and capable of connecting the opening of the housing and the opening of the cultivation container; a first lid portion closing one open end of the connection hole; and a second lid portion closing the other open end of the connection hole. The first and second lid portion defines there-between a sealed space in the connection hole. The sealed space is capable of containing therein a second solution liquid for the cell culture.

### CROSS REFERENCE TO RELATED APPLICATIONS

This application claims priority based on 35 USC 119 from prior Japanese Patent Application No. 2008-328108 filed on Dec. 24, 2008, entitled “automatic culture device, sealed container, slide valve and disposable container”, the entire contents of which are incorporated herein by reference.

### BACKGROUND OF THE INVENTION

1. Field of the Invention

The invention relates to an automatic culture device, a slide valve and a disposable container used for cell culture.

2. Description of Related Art

Cell culture for proliferating or differentiating cells has been widely used. Such a cell culture includes complex steps, that is, a step of culturing a cell with a culture medium, a step of washing the cultured cells with PBS (Phosphate Buffered Salts), a step of peeling the cultured cells off a cultivation container by using trypsin, and a step of adding different reagents, and the like.

An automatic culture device for performing cell culture automatically is proposed (See, Japanese Patent Application Laid-Open No. 2007-3126668). Specifically, the automatic culture device includes a cultivation container for cultivating a cell therein, reagent containers respectively reserving therein different reagents such as PBS, trypsin or the like, tubes connecting the cultivation container to the reagent containers, a pump configured to send the reagents from the reagent containers to the cultivation container via the tubes, and valves that are respectively provided at the tubes and configured to open and close the tubes. The reagents are supplied respectively from the reagent containers to the cultivation container in a particular order.

### SUMMARY OF THE INVENTION

The automatic culture device disclosed in Japanese Patent

Application Laid-Open No. 2007-3126668 has the following problems. First, the cultivation container, the reagent containers, and the tubes all must be thrown away after the cell culture. The many tubes must be thrown away and large amounts of the reagents remain in the many tubes that must be thrown away, and this increases the cost of the cell culture. Second, since plural types of reagents are used for the cell culture, a complex control of switching passages (the tubes) for supplying the reagents through the tubes is needed.

A first aspect of the invention is an automatic culture device including: a housing accommodating therein a preservation container preserving a first solution liquid for cell culture, the housing having an opening for discharging the first solution liquid out of the housing; a cultivation container in which cell culture can be performed and having an opening; and a slide valve provided between the housing and the cultivation container. The slide valve includes: a slide valve body; a connection hole formed in the slide valve body and capable of connecting the opening of the housing and the opening of the cultivation container; a first lid portion closing one of open ends of the connection hole which is provided on the housing side; and a second lid portion closing the other open end of the connection hole that is provided on the cultivation container side. The first and second lid portions define there-between a sealed space in the connection hole. The sealed space is capable of containing therein a second solution liquid used for the cell culture with the first solution liquid.

In the automatic culture device of the first aspect, the first lid portion and the second lid portion may be made of a resin thin film. The first lid portion and/or the second lid portion may be a rigid body capable of being opened by inserting into the connection hole a pipe attached to the opening of the housing or the opening of cultivation container, or capable of being opened by catching the rigid body with a projection formed at the housing or the cultivation container as the slide valve is slid.

In the automatic culture device of the first aspect, the opening of the cultivation container may be an opening formed at a lid closing an opening of the cultivation container and may be an upper opening of the cultivation container that does not have a lid.

In the automatic culture device of the first aspect, the second solution liquid may be sealed in the sealed space.

In the automatic culture device of the first aspect, the slide valve body may be formed with a through hole that connects the sealed space and the outside of the slide valve body.

An second aspect of the invention is a sealed container including: a tubular member capable of being fixed in a connection hole of a slide valve, the connection hole configured to connect, in a position of the slide valve, an opening of a housing for discharging a first solution liquid for cell culture and an opening of a cultivation container configured to perform cell culture therein; a first lid portion closing one of the open ends of the tubular member, and a second lid portion closing the other open end of the tubular member, wherein the first lid portion and the second lid portion define a sealed space between the first lid portion and the second lid portion in the tubular member, the sealed space being capable of sealing therein a second solution liquid used for cell culture with the first solution liquid.

An third aspect of the invention is a slide valve including: a slide valve body capable of being provided between a housing accommodating therein a preservation container preserving a first solution liquid for cell culture and a cultivation container configured to perform cell culture therein; a connection hole formed in the slide body, the connection hole configured to connect, in a position of the slide valve body, an opening of the housing for discharging the first solution liquid and an opening of the cultivation container; a first lid portion closing one of the open ends of the connection hole, and a second lid portion closing the other open end of the connection hole. The first lid portion and the second lid portion define a sealed space there-between in the connection hole. The sealed space is capable of sealing therein a second solution liquid used for cell culture with the first solution liquid.

An fourth aspect of the invention is a disposable container including: a housing accommodating therein a preservation container preserving a first solution liquid for cell culture, the housing having an opening for discharging the first solution liquid out of the housing; and a slide valve capable of being slid with respect to the housing. The slide valve including: a slide valve body; a connection hole formed in the slide valve body, the connection hole configured to communicate with the opening of the housing, in a slide position of the slide valve body; a first lid portion closing one of the open ends of the connection hole; and a second lid portion closing the other open end of the connection hole. The first and second lid portions define there-between a sealed space in the connection hole. The sealed space is capable of sealing therein a second solution liquid used for the cell culture with the first solution liquid.

The aspects of the invention eliminate the complex control and reduce the cost of the cell culture.

### BRIEF DESCRIPTION OF THE DRAWINGS

FIG. 1 is a side view of a configuration of an automatic culture device 100 according to a first embodiment of the invention.

FIG. 2 is a view showing a configuration of slide valve 20 according to the first embodiment of the invention.

FIG. 3 is a view showing a configuration of slide valve 20 according to a second embodiment of the invention.

FIG. 4 is a view showing a configuration of slide valve 20 according to a third embodiment.

FIG. 5 is a sectional view of sealed container 60 according to the third embodiment of the invention.

FIG. 6 is a view showing a configuration of slide valve 20 according to another embodiment of the invention.

### DETAILED DESCRIPTION OF EMBODIMENTS

Next, embodiments of the invention will be described with reference to the drawings. In the respective drawings referenced herein, the same constituents are designated by the same reference numerals and duplicate explanation concerning the same constituents is basically omitted. All of the drawings are provided to illustrate the respective examples only. No dimensional proportions in the drawings shall impose a restriction on the embodiments. For this reason, specific dimensions and the like should be interpreted with the following descriptions taken into consideration. In addition, the drawings include parts whose dimensional relationship and ratios are different from one drawing to another.

### First Embodiment

### Configuration of Automatic Culture Device

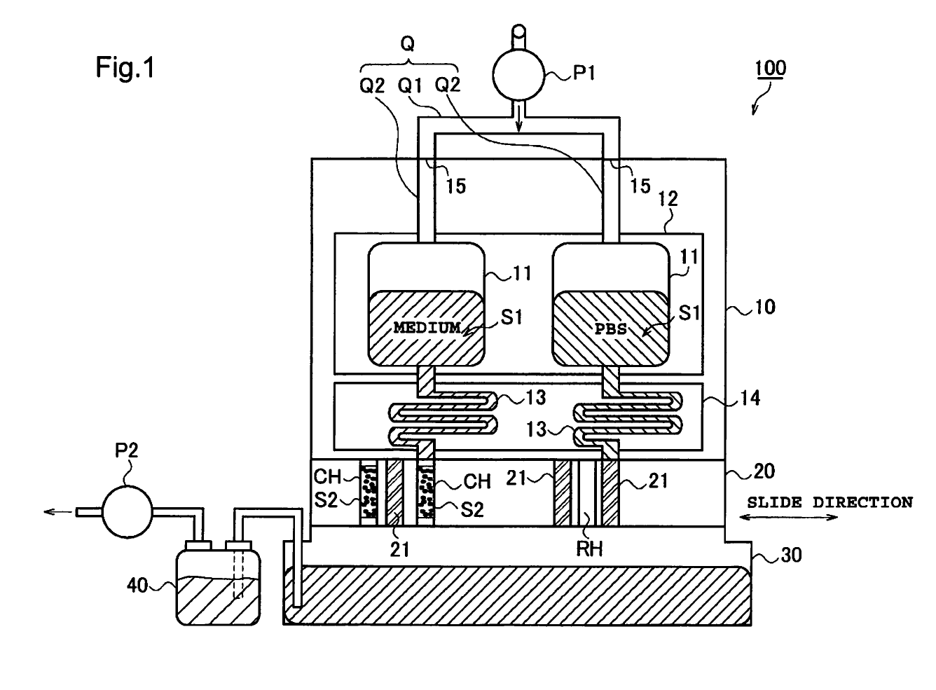

A configuration of an automatic culture device of a first embodiment will be described with reference to FIG. 1. FIG. 1 is a side view of a configuration of automatic culture device 100 according to the first embodiment.

As shown in FIG. 1, automatic culture device 100 includes housing 10, slide valve 20, cultivation container 30 (for example, dish, flask, microplate, bottle, etc.), waste tank 40, pump P1, air line Q, and pump P2. Automatic culture device 100 is a device for automatically performing cell culture such as a cell proliferation, a cell differentiation, or the like. A cell culture means culturing a cell obtained from a human body in quasi-intercellular fluid (hereinafter referred to as “culture medium”). Automatic culture device 100 is categorized into two types: a sealed container type wherein the automatic culture device has a sealed container therein; a housing sealing type wherein the housing sealingly accommodates an automatic culture device therein.

Housing 10 accommodates therein preservation containers 11, cooling unit 12, pipes 13 and heating unit 14.

Preservation containers 11 preserve therein first solution liquids S1 (such as a culture medium, PBS or the like) for cell culture, respectively. Preservation containers 11 are cooled by cooling unit 12 so that each of first solution liquids S1 is kept cooled to an appropriate temperature. That is, first solution liquids S1 are preserved in preservation containers 11 in good condition. Each preservation container 11 has an outlet. The outlets of the preservation containers 11 are connected to openings (culture medium outlet and PBS outlet in this embodiment) of housing 10 via pipes 13.

Pipes 13 connect the outlets of preservation containers 11 with slide valve 20. First solution liquids S1 flow from preservation containers 11 to slide valve 20 through pipes 13. Pipes 13 are heated by heating unit 14 to a certain temperature such as a temperature of the human body (about 37° C.). First solution liquids S1 are thus supplied to cultivation container 30 with an appropriate temperature for the cell culture in cultivation container 30. Note that, automatic culture device 100 may not have heating unit 14 if first solution liquids S1 do not need to be heated.

Housing 10 includes through holes 15 through which air lines Q are inserted. Through air line Q, air is pumped from pump P1 to preservation containers 11. Air lines Q includes first air line Q1 and second air line Q2. First air line Q1 is provided outside housing 10 and connects pump P1 with housing 10. Second air line Q2 is provided inside housing 10 and connects first air line Q1 with preservation containers 11. In this embodiment, first air line Q1 is formed separately with and is connected with second air lines Q2 at through holes 15. Note that first air line Q1 and second air line Q2 may be formed integrally with each other to be a one-peace product.

Slide valve 20 is provided between housing 10 and cultivation container 30 and separates them. As shown in FIG. 1, slide valve 20 includes slide valve body H, stoppers 21 provided at slide valve body H, communication hole RH and connection holes CH formed through slide valve body H. Communication hole RH is a passage in which one of first solution liquids S1 (“PBS” in this embodiment) flows. Second solution liquids S2 (for example, reagent such as blood serum, growth factor or stimulation factor) are sealed in connection holes CH, respectively. Second solution liquids S2 are used for cell culture with the other of first solution liquids S1 (“culture medium” in this embodiment). Slide valve 20 is slidable in a slide direction shown in FIG. 1 and supply of first solution liquids S1 and/or second solution liquids S2 is controlled by slide positions of slide valve 20.

For example, upon supplying culture medium S1 serving as a first solution liquid S1 with second solution liquid S2 to cultivation container 30, slide valve 20 is slid to a slide position where connection hole CH of slide valve 20 and the culture medium outlet of housing 10 (which is the open end of culture medium pipe 13 in this embodiment) are aligned with each other. At this slide position, stopper 21 of slide valve 20 is in contact with and closes the PBS outlet of housing 10 (the opening end of PBS pipe 13). Upon supplying PBS serving as a first solution liquid S1 to cultivation container 30, slide valve 20 is slid to a slide position where the PBS outlet of housing 10 and communication hole RH are aligned with each other. At this slide position, stopper 21 of slide valve 20 is in contact with and closes the culture medium outlet of housing 10.

This embodiment thus can supply culture medium S1 and PBS S1 separately and can supply culture medium S1 and second solution liquid S2 together, according to slide positions of slide valve 20. Note that slide valve 20 may have such a configuration that more than two types of first solution liquids can be supplied at the same time. The configuration of slide valve 20 will be described in detail later. Note that reagents such as blood serum, growth factor, stimulation factor or the like are very expensive compared to culture medium or the like in general.

Cultivation container 30 forms therein a field to perform cell culture. First solution liquids S1 and second solution liquids S2 are supplied to cultivation container 30 through an opening of cultivation container 30 by using slide valve 20. If cultivation container 30 includes a lid, the opening of cultivation container 30 is a hole formed at the lid. If cultivation container 30 does not include a lid to close an upper opening, the opening of cultivation container is the upper opening. The inside space of cultivation container 30 is controlled to have an atmosphere (for example, CO2 gas at 37° C.) suited for the cell culture. When the cell culture is finished, first solution liquids S1 and second solution liquids S2 that have been supplied to cultivation container 30 are discharged to waste tank 40.

Upon supplying first solution liquids S1 and/or second solution liquids S2 to cultivation container 30, pump P1 pumps air into preservation containers 11 so that first solution liquids S1 are forced out of preservation containers 11 to pipes 13 and to cultivation container 30. Note that, instead of pump P1, other type pumps, for example, a perista pump or the like may be used as a liquid transmitting unit to force first solution liquids S1 out of preservation container 11 into pipes 13. In this case (not shown), the perista pump may be attached to pipes 13 to squeeze first solution liquids S1 from preservation containers 11 toward cultivation container 30.

Upon discharging first solution liquids S1 and second solution liquids S2 from cultivation container 30 to waste tank 40, pump P2 sucks out air from waste tank 40 so that first solution liquids S1 and second solution liquids S2 are discharged from cultivation container 30 to waste tank 40. Note that, other type pump, for example, a perista pump or the like may be used instead of pump P2.

Preservation containers 11, pipes 13, and slide valve 20 are disposed of, after finishing the cell culture. Upon performing a next cell culture, new preservation container 11, new pipes 13 and new slide valve 20 will be used. To prevent contamination, housing 10 (including preservation containers 11, pipes 13 and second air line Q2) and slide valve 20 constitute a disposable container of automatic culture device 100. The disposable container is preferably made of resin material such as polypropylene or the like. Note that second air lines Q2 in the disposal container serve as an external force applying mechanism for applying an external force to first solution liquids S. In case of using a perista pump instead of pump P1, rollers of the perista pump attached to pipes 13 or the like serve as an external force applying mechanism. Although the disposable container is provided above cultivation container 30 and does not includes cultivation container 30 in this embodiment, the disposable container may include cultivation container 30 in the invention.

Automatic culture device 100 may include a cooling unit (not shown) to cool slide valve 20 so that second solution liquids S2 that is sealed in slide valve 20 can be preserved in a good condition.

### Detail Configuration of Slide Valve

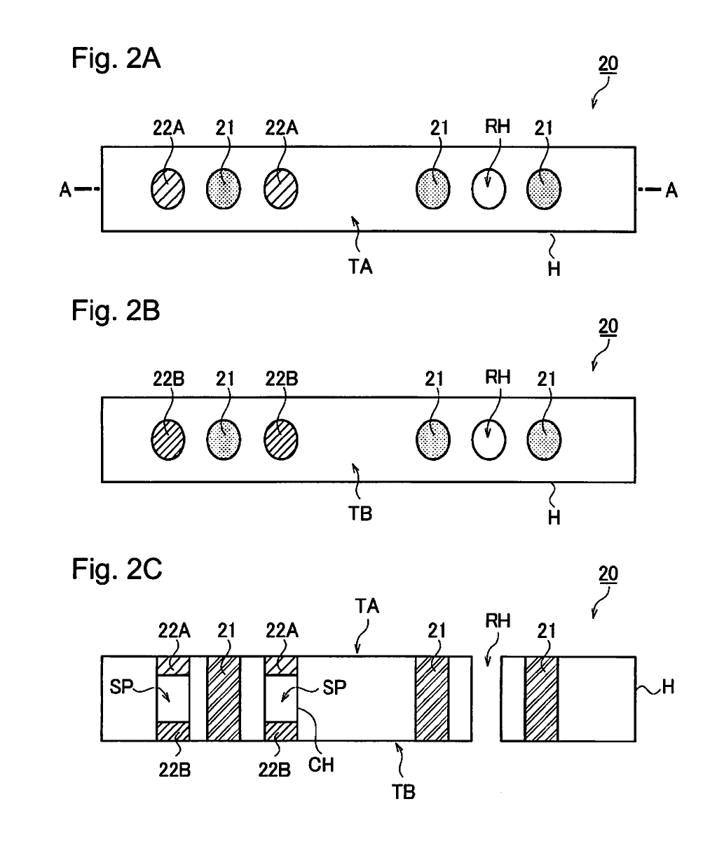

The detail configuration of slide valve 20 according to the first embodiment will be described with reference to FIGS. 2A, 2B and 2c. FIG. 2A is a plan view of slide valve 20 as seen from housing 10. FIG. 2B is a plan view of slide valve 20 as seen from cultivation container 30. FIG. 2C is a sectional view along A-A line in FIG. 2A.

As shown in FIG. 2, slide valve 20 includes slide valve body H, stoppers 21, communication hole RH, connection hole CH, first lid portion 22A, second lid portion 22B and sealed space SP.

Slide valve body H is formed with first opposed surface TA opposed to housing 10 and second opposed surface TB opposed to cultivation container 30. As describe above, slide valve body H is preferably made of resin material, however may be made of other material such as glass, ceramic, or the like.

Stoppers 21 are capable of closing or covering the lower opening ends of pipes 13 so as to stop leaking first solution liquids S1 out of pipes 13. To prevent supply of first solution liquids S1 from preservation containers 11 to cultivation container 30, slide valve is slid to a position where stoppers 21 are aligned with the lower open ends of pipes 13. Each stopper 21 is made of rubber or the like. Each stopper 21 extends through slide valve body H from first opposed surface TA to second opposed surface TB in this embodiment. However, stopper 21 may be provided only at first opposed surface TA of slide valve body H and is not required to extend from first opposed surface TA to second opposed surface TB.

Communication hole RH is formed in slide valve body H and connects and communicates the PBS outlet of housing 10 with the opening of cultivation container 30 at a slide position of slide valve 20. Communication hole RH is a passage that extends through slide valve body H from first opposed surface TA to second opposed surface TB.

Connection hole CH is formed in slide valve body H and capable of connecting and communicating the culture medium outlet of housing 10 with the opening of cultivation container 30 at a slide position of slide valve 20. Connection hole CH is a passage that extends through slide valve body H from first opposed surface TA to second opposed surface TB.

First lid portion 22A closes one of the open ends of connection hole CH that is provided on the housing side. Second lid portion 22B closes the other open end of connection hole CH that is provided on the cultivation container side. First lid portion 22A and second lid portion 22B are formed of thin resin film and capable of being torn opened by pressure in this embodiment. Lid portions 22A and/or 22B are not limited to the above described configurations and materials. For example, lid portions 22A and/or 22B may be a rigid body capable of being opened by inserting a pipe attached to the outlets of housing 10 or the opening of cultivation container 30 into the rigid body, or capable of being slidably or rotatably opened by catching the rigid body with a projection (not shown) formed at housing 10 or cultivation container 30 as slide valve 20 is slid.

Sealed space SP is defined between first lid portion 22A and second lid portion 22B in connection hole CH and is capable of sealingly containing therein second solution liquid S2. In this embodiment, second solution liquid S2 is sealed in sealed space SP.

### Operation of Supplying Solution Liquids

Next, an operation for supplying solution liquids from housing 10 to cultivation container 30 via slide valve 20 will be described. Note that the following will be described, assuming that first lid portion 22A and second lid portion 22B are thin resin films configured to be torn by pressure. First lid portion 22A may be capable of being torn by a lower pressure than second lid portion 22B in this example.

First, to supply culture medium to cultivation container 30, slide valve 20 is slid along the slide direction shown in FIG. 1 to a position where first lid portion 22A is aligned with the lower opening end of culture medium pipe 13.

Next, pump P1 is activated so that culture medium S1 is forced out of the lower opening end of culture medium pipe 13 to tear first lid portion 22A. With this operation, culture medium S1 goes into sealed space SP and thus culture medium S1 and second solution liquid S2 are mixed with each other.

Next, by using pump P1, culture medium S1 is further forced out of the lower open end of culture medium pipe 13, so as to tear second lid portion 22B. With this operation, culture medium S1 and second solution liquid S2 are supplied into cultivation container 30 together.

### Effects

Automatic culture device 100 of the first embodiment has slide valve 20 provided between housing 10 and cultivation container 30. Slide valve 20 has sealed space SP that is defined between first lid portion 22A and second lid portion 22B in connection hole CH and is capable of sealing second solution liquid S2 therein.

According to the first embodiment, second solution liquid S2 is sealed in slide valve 20, thereby reducing the amount of second solution liquid S2 that will be disposed of. This brings about reduced cost for the cell culture. Since second solution liquids S2 are generally expensive reagents such as blood serum, growth factor, stimulation factor or the like, automatic culture device 100 has a big cost reduction effect.

Without a preservation container for second solution liquid and without a pipe for connecting cultivation container 30 and the preservation container for second solution liquid, this embodiment can supply first and second solution liquids S1 and S2 from housing 10 to cultivation container 30. This reduces the number of parts to be disposed of in order to prevent contamination, thereby further reducing the cost for the cell culture.

Further, this embodiment can control the supply of many different type solution liquids including first solution liquid S1 and second solution liquid S2, just by controlling the slide position of slide valve 20. That is, this embodiment does not need a complex control of pumps and valves for switching between passages for solutions, upon supplying first solution liquid S1 and second solution liquid S2.

### Second Embodiment

Next, a second embodiment of the invention will be described with reference to the drawings. The following explanation will be made mainly about differences between the second embodiment and the first embodiment. Briefly, slide valve 20 according to the second embodiment has such a configuration that second solution liquid S2 can be filled into sealed space SP when needed.

### Configuration of Slide Valve

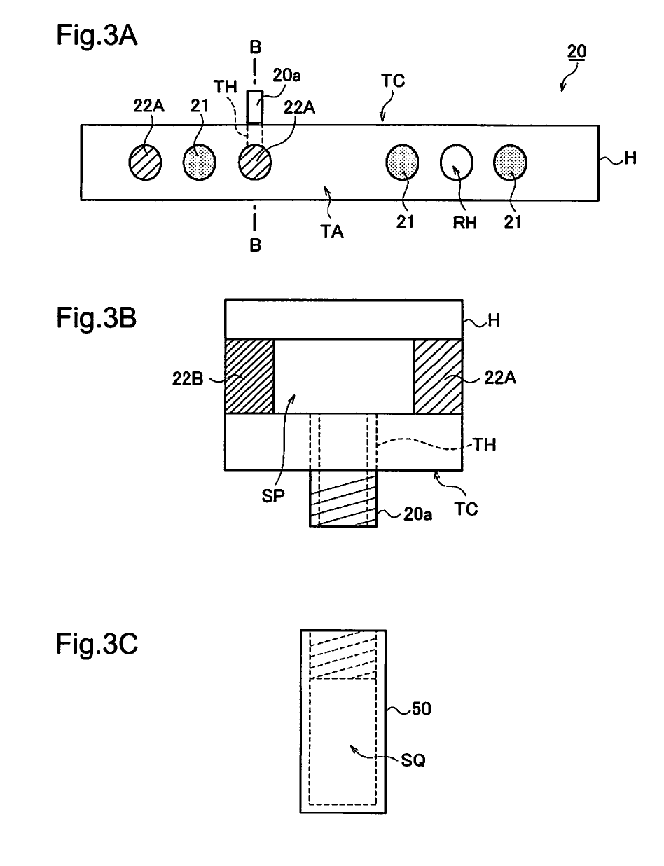

A configuration of slide valve 20 according to the second embodiment will be described with reference to FIG. 3. FIG. 3A is a plan view of slide valve 20 as seen from housing 10. FIG. 3B is a sectional view of slide valve 20 along B-B line in FIG. 3A. FIG. 3C is a view showing vial 50 to be attached to slide valve 20.

As shown in FIGS. 3A and 3B, slide valve body H of slide valve 20 is formed with through hole TH and projection 20a in addition to the configuration of the slide valve body H described in the first embodiment.

Through hole TH is formed separately from connection hole CH and extends from side surface TC of slide valve body H to sealed space SP to connect and communicate sealed space SP with the outside of slide valve body H. Projection 20a is formed in a tubular shape such that the inside space of projection 20a continues through hole TH. An outer circumferential surface of projection 20a has a thread groove such that projection 20a functions as a male thread.

Vial 50 is a sealed vessel containing therein second solution liquid S2. As shown in FIG. 3C, vial 50 forms therein sealed space SQ to seal therein second solution liquid S2. An inner circumferential surface of vial 50 has a thread groove which is fit to the thread groove of projection 20a, such that vial 50 functions as a female thread with respect to projection 20a serving as the male thread. Note that when vial 50 is transported (that is, before using vial 50), vial 50 is closed with a threaded lid (not shown).

### Operation of Supplying Solution Liquids

First, the lid is unfastened and removed from vial 50, and then vial 50 is screwed to projection 20a of slide valve 20. Next, slide valve 20 is placed between housing 10 and cultivation container 30.

Next, to supply culture medium S1 to cultivation container 30, slide valve 20 is slid along the slide direction to a position where first lid portion 22A is aligned with the lower opening end of culture medium pipe 13.

Next, by using pump P1, culture medium S1 is forced out of the lower opening end of culture medium pipe 13 so as to tear first lid portion 22A. With this operation, culture medium S1 flows into sealed space SP and thus culture medium S1 and second solution liquid S2 are mixed with each other.

Next, by using pump P1, culture medium S1 is further forced out of the lower opening end of culture medium pipe 13 so as to tear second lid portion 22B. With this operation, culture medium S1 and second solution liquid S2 are supplied into cultivation container 30 together.

### Effect

Slide valve 20 according to the second embodiment includes through hole TH which extends from side surface H of slide valve body H to sealed space SP of slide valve body H. Therefore, second solution liquid S2 can be filled into sealed space SP of slide valve 20 at the time when slide valve 20 is used. In other words, second solution liquid S2 can be separately preserved from slide valve 20. This improves handling ability of second solution liquid S2.

### Third Embodiment

Next, a third embodiment of the invention will be described with reference to the drawings. The following explanation will be made mainly about differences between the third embodiment and the first embodiment. Briefly, slide valve 20 according to the third embodiment has a sealed container. The sealed container seals therein second solution liquid S2 and is capable of being fixed in connection hole CH formed through slide valve body H.

### Configuration of Slide Valve

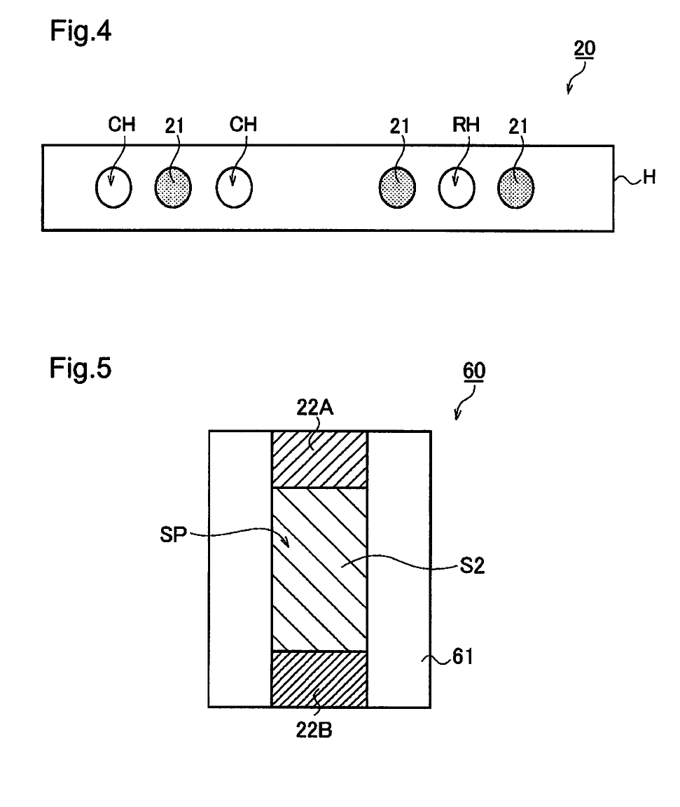

A configuration of slide valve 20 according to the third embodiment will be described with reference to FIG. 4. FIG. 4 is a plan view of slide valve 20 as seen from housing 10. As shown in FIG. 4, slide valve 20 does not have first lid portion 22A and second lid portion 22B that are directly attached to and close connection hole CH. That is, slide valve 20 does not have sealed space SP until sealed container 60 is fixed in connection hole CH of slide valve body H.

### Configuration of Sealed Container

Configuration of sealed container 60 according to the third embodiment will be described with reference to FIG. 5. FIG. 5 is a sectional view of sealed container 60.

As shown in FIG. 5, sealed container 60 includes tubular member 61, first lid portion 22A, second lid portion 22B and sealed space SP.

Tubular member 61 is formed in such a shape that an outer circumferential surface thereof is to be fit in inner circumferential surface of connection hole CH. Tubular member 61 has therein a through hole.

First lid portion 22A is attached to and closes one of opposite open ends of the through hole of tubular member 61. Second lid portion 22B is attached to and closes the other of opposite open ends of the through hole of tubular member 61. Therefore, sealed space SP is formed between first lid portion 22A and second lid portion 22B in the through hole of tubular member 61. Second solution liquid S2 is filled in sealed space SP of sealed container 60.

### Supply of Solution Liquids

First, sealed container 60 is inserted into and fixed to connection hole CH of slide valve 20. Next, slide valve 20 is placed between housing 10 and cultivation container 30.

Next, operation of supplying solution liquids from housing 10 through slide valve 20 to cultivation container 3 will be described.

First, upon supplying culture medium S1 serving as a first solution liquid and second solution liquid S2 to cultivation container 30, slide valve 20 is slid along the slide direction to a position where first lid portion 22A is aligned with the lower open end of culture medium pipe 13.

Next, by using pump P1, culture medium S1 is forced out of the lower opening end of culture medium pipe 13, so as to tear first lid portion 22A. With this operation, culture medium S1 flows into sealed space SP and then culture medium S1 and second solution liquid S2 are mixed with each other.

Next, by using pump P1, culture medium S1 is further forced out of the lower open end of culture medium pipe 13, so as to tear first lid portion 22B. With this operation, culture medium S1 and second solution liquid S2 are supplied into cultivation container 30 together.

### Effects

According to the third embodiment, sealed container 60 seals second solution liquid S2 in sealed space SP thereof and is fixed in connection hole CH of slide valve 20.

Therefore, second solution liquid S2 is conserved separately from slide valve 20, and this improves handling ability of second solution liquid S2.

### Other Embodiments

The invention has been described with reference to the above embodiments; however, it should be appreciated that the invention is not limited to the above embodiments. Various changes and modifications of embodiments, examples and operations will be apparent to persons skilled in the art based on the above disclosure.

For example, although vial 50 is attached to through hole TH in the second embodiment, through hole TH may be sealed by a rubber lid or the like so that second solution liquid S2 can be injected into sealed space SP by an injection at the time when slide valve 20 is used.

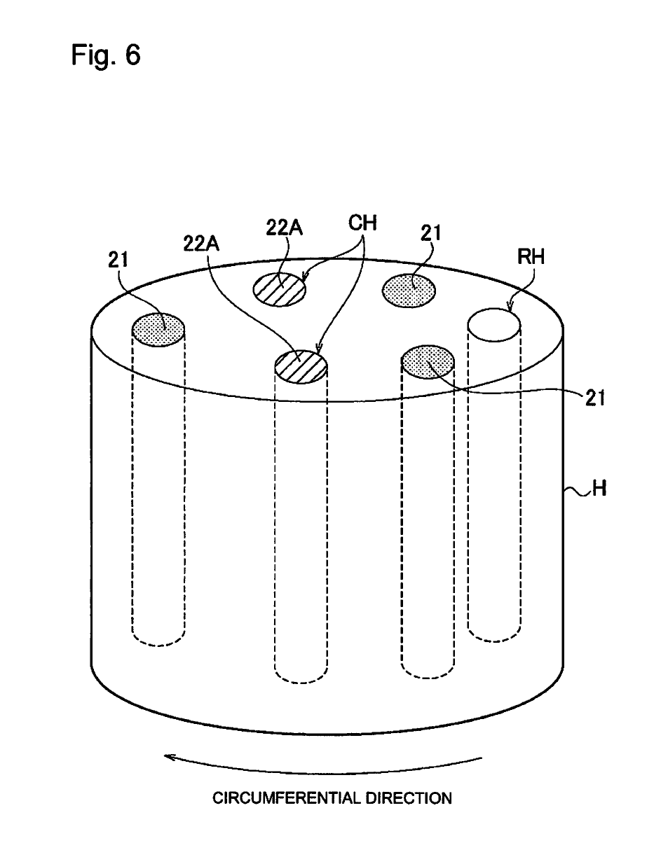

Although the above embodiments have slide valve 20 capable of linearly sliding in one direction, the invention is not limited to this manner. For example, as shown in FIG. 6, slide valve body H may be formed in a cylindrical column shape with stopper 21, communication hole RH, and connection hole CH. Such a cylindrical slide valve 20 is slid in a circumferential direction thereby controlling supply of first solution liquids S1 and second solution liquids S2. It should be understood that slide valve 20 includes a valve that can control the supply of solution liquids by a “movement” including slide movement, rotation movement or the like,

Further, although automatic culture device 100 includes pump P1 and pump P2 serving as a liquid transmitting unit in the above embodiments, the invention is not limited to this. For example, automatic culture device 100 may include a known liquid transmitting unit such as a syringe pump, perista pump or the like as a liquid transmitting unit instead of pump P1.

It should be understood that the invention includes various embodiments and the like that are not described above. Therefore, the invention is limited only by the subject matter defined in the appended claims in the application.

What is claimed is:

1. An automatic culture device comprising:
a housing accommodating therein a preservation container preserving a first solution liquid for cell culture, the housing having an opening for discharging the first solution liquid out of the housing;
a cultivation container in which the cell culture can be performed and having an opening; and
a slide valve provided between the housing and the cultivation container,
the slide valve comprising:
a slide valve body;
a connection hole formed in the slide valve body and capable of connecting the opening of the housing and the opening of the cultivation container;
a first lid portion closing one of open ends of the connection hole that is on the housing side; and
a second lid portion closing the other of the open ends of the connection hole that is on the cultivation container side, the first and second lid portions defining there-between a sealed space in the connection hole, the sealed space being capable of containing therein a second solution liquid used for the cell culture with the first solution liquid.

2. The automatic culture device of claim 1, further comprising:
an opener configured to open the first lid portion and/or the second lid portion.

3. The automatic culture device of claim 2, wherein
the opener is a liquid transmitting unit configured to force the first solution liquid out of the preservation container toward the cultivation container, which can open the first lid portion and/or the second lid portion by pressure.

4. The automatic culture device of claim 2, wherein
the opener is a mechanical engagement which opens the first lid portion and/or the second lid portion as the slide valve is slid.

5. The automatic culture device according to claim 1, wherein:
the second solution liquid is sealed in the sealed space.

6. The automatic culture device according to claim 1, wherein:
the slide valve body is formed with a through hole communicating the sealed space of the connection hole with outside of the slide valve body.

7. The automatic culture device according to claim 1, wherein:
the slide valve is sandwiched between the housing and the cultivation container such that the opening of the housing and the one of the open ends of the connection hole directly contact with each other and the opening of the cultivation container and the other of the open ends of the connection hole directly contact with each other.

8. The automatic culture device according to claim 1, wherein:
the housing is located above the cultivation container.

9. The automatic culture device according to claim 1, wherein:
the sealed space is defined by the inner circumferential surface of the connection hole, the first lid portion, and the second lid portion.

10. The automatic culture device according to claim 1, wherein:
the sealed space is a space defined by a tubular member being fit to the inner circumferential surface of the connection hole, the first lid portion closing one of the open ends of the tubular member, and the second lid portion closing the other of the open ends of the tubular member.

11. The automatic culture device according to claim 1, wherein:
the first lid portion is able to be opened by a lower pressure than the second lid portion.

12. A slide valve, comprising:
a slide valve body capable of being provided between a housing accommodating therein a preservation container preserving a first solution liquid for cell culture and a cultivation container configured to perform the cell culture therein;
a connection hole formed in the slide body, the connection hole configured to connect, in a position of the slide valve body, an opening of the housing for discharging the first solution liquid and an opening of the cultivation container;
a first lid portion closing one of the open ends of the connection hole; and
a second lid portion closing the other of the open ends of the connection hole, wherein the first lid portion and the second lid portion define a sealed space between the first lid portion and the second lid portion in the connection hole, and the sealed space is capable of sealing therein a second solution liquid used for the cell culture with the first solution liquid.

13. A disposable container, comprising:
a housing accommodating therein a preservation container preserving a first solution liquid for cell culture, the housing having an opening for discharging the first solution liquid out of the housing; and
a slide valve capable of being slid with respect to the housing;
the slide valve comprising:
a slide valve body;
a connection hole formed in the slide valve body, the connection hole configured to communicate with the opening of the housing, in a slide position of the slide valve body;
a first lid portion closing one of the open ends of the connection hole; and
a second lid portion closing the other of the open ends of the connection hole, the first and second lid portion defining there-between a sealed space in the connection hole, the sealed space being capable of containing therein a second solution liquid used for the cell culture with the first solution liquid.

14. The disposable container according to claim 13, wherein
the housing includes an external force applying mechanism configured to force the first solution liquid out of the preservation container toward the opening of the housing.

15. The disposable container according to claim 14, wherein:
the external force applying mechanism is a pipe for connecting a pump and the preservation container, the pipe allows pressure to be applied from the pump into the preservation container so that the first solution liquid is forced out of the preservation container toward the opening of the housing.

16. The disposable container according to claim 14, further comprising:
a pipe connecting an outlet of the preservation container for discharging the first solution liquid and the opening of the housing,
wherein the external force applying mechanism is a pump attached to the pipe.
